(19) United States
(12) Patent Application Publication
(10) Pub. No.: US 2025/0381006 A1
(43) Pub. Date: Dec. 18, 2025

(54) SUTURE MEASURING DEVICES AND METHODS

(71) Applicant: Cor Medical Ventures, Inc., Del Mar, CA (US)

(72) Inventors: Brian Bowman, Carlsbad, CA (US); Benjamin Arnold, San Diego, CA (US)

(21) Appl. No.: 19/236,888

(22) Filed: Jun. 12, 2025

Related U.S. Application Data
(60) Provisional application No. 63/659,120, filed on Jun. 12, 2024.

(51) Int. Cl. A61B 90/00; A61B 17/00; A61B 17/06

(52) U.S. Cl. CPC A61B 90/06; A61B 17/06166; A61B 2017/00526; A61B 2090/061; A61B 2090/3937; A61B 2090/3966

(57) ABSTRACT

Suture systems, devices, and methods to enhance the precision and efficiency of surgical procedures are disclosed herein. Exemplary sutures are integrated with length indicators along their length, providing surgeons with an immediate visual reference for measuring anatomical distances intra-operatively in open and key hole (e.g. arthroscopic and endoscopic) procedures, tissue dimensions, gauging suture tension, and determining the optimal or desired suture length required for closure. The length indicators, marked at predetermined intervals, allow for accurate measurement without the need for additional tools, thereby streamlining the surgical process and reducing operation time. This suture material is particularly beneficial in complex surgeries where precision is paramount, and can improve patient outcomes by enabling more accurate tissue approximation and wound closure. Exemplary embodiments enable a combination of functionality and convenience, providing a valuable addition to the medical field, particularly in surgical specialties that demand meticulous attention to detail.

### CROSS-REFERENCES TO RELATED APPLICATIONS

This application claims the benefit of U.S. Provisional Patent Application No. 63/659,120 filed Jun. 12, 2024, the disclosure of which is incorporated herein by reference.

### BACKGROUND

Embodiments of the present invention relate generally to suture devices and methods, and in particular instances, to suture measuring devices and methods the enable use of a suture device as a ruler or measurement means.

Sutures are specialized medical devices used for wound closure and soft tissue fixation during or after surgical procedures. They play a crucial role in facilitating the healing process by approximating the edges of a wound or incision to ensure it stays closed. Sutures are made from a variety of materials, both natural and synthetic, which can be absorbable or nonabsorbable. Absorbable sutures are designed to be broken down by the body over time and do not require removal, making them ideal for internal tissues. Nonabsorbable sutures, on the other hand, either need to be removed after a certain period or are left in place permanently, depending on the location and purpose of the suture.

Sutures are made from a variety of materials, each chosen for its specific properties and suitability for different types of surgical procedures. Historically, sutures were crafted from natural fibers such as silk, linen, and the intestines of cows or sheep, known as catgut. These materials were prized for their strength and ease of use. However, with advances in technology, synthetic materials have become more prevalent in suture manufacturing. Modern sutures are often made from synthetic polymers like nylon, polyester, polypropylene, polyethylene (UHMWPE) and polyglycolic acid, which can be engineered to possess a range of properties, such as tensile strength and controlled rates of absorption by the body.

The choice between absorbable and non-absorbable sutures depends on the needs of the surgical procedure. Absorbable sutures, such as those made from polyglycolic acid or polylactic acid, are designed to be broken down by the body over time, eliminating the need for removal and are often used for internal tissues that heal quickly. Non-absorbable sutures, on the other hand, are made from materials like silk, nylon, or polypropylene, UHMWPE and are either removed after a certain period or left in permanently for tissues that require longer-term support.

In addition to the basic materials, sutures can also be coated with substances to enhance their performance. Coatings can be applied to reduce friction during suturing, to provide antibacterial properties, or to promote healing. For example, some sutures are coated with a substance that triggers the body's natural healing processes, thereby reducing inflammation and scarring.

In addition to material composition, sutures can be classified based on their structure. Monofilament sutures consist of a single thread, allowing for smooth passage through tissues, while braided sutures are made of several small threads intertwined. The technique of suturing also varies, with different methods employed to optimize healing and minimize scarring. Proper suture technique is crucial for wound healing, as it affects the tension distributed across the wound, the risk of infection, and the final appearance of the healed tissue.

Sutures are not only used for closing wounds but also for securing medical devices, like drains or tubes, to the body, and for ligating blood vessels during surgery to prevent bleeding. Suture may also be used for the repair of soft tissue injuries, which are common in sports-related activities. The use of sutures provides a means to reattach torn ligaments and tendons to their corresponding bones, ensuring a secure and precise alignment that is crucial for proper healing.

Many surgical procedures are completed minimally invasively where limited access to the surgical site is available. Arthroscopic and laparoscopic surgeries are minimally invasive procedures that require small instruments due to the nature of the techniques used. These surgeries are performed through tiny incisions, typically only a few millimeters long, which significantly reduces the damage to the surrounding tissues compared to traditional open surgeries. The use of small, precise instruments allows surgeons to navigate these incisions and perform complex procedures within confined spaces inside the body. For instance, in arthroscopy, which is often used for joint surgeries, the arthroscope and surgical instruments are thin, enabling the surgeon to minimize the size of the incisions. This results in less postoperative pain and joint stiffness, and often shortens the recovery time, allowing patients to return to their activities more quickly.

Similarly, laparoscopic surgery involves the use of specialized instruments, including a laparoscope (e.g. a thin, telescopic rod with a video camera on the end) to view and operate on the internal organs through small abdominal incisions. The camera projects an image of the internal area onto a monitor, giving the surgeon a clear view without the need for a large incision. The smaller instruments used in laparoscopy lead to less cutting, reduced risk of infection, and less postoperative pain, contributing to a quicker recovery and better overall patient outcomes. These advancements in surgical technology and technique have revolutionized many aspects of surgery, offering safer options with improved recovery times for patients.

Current suture modalities enable surgeons to provide beneficial treatments to patients in need thereof. Yet still further improvements in suture technology are desired.

Embodiments of the present invention provide solutions to at least some of these outstanding needs.

For example, suture measuring devices disclosed herein can be ideal for minimally invasive surgeries where traditional measuring instruments are too large, or where other methods for measuring distances in minimally invasive surgeries are too complex or too expensive.

### BRIEF SUMMARY

Exemplary suture measuring devices disclosed herein may include a flexible elongate member (which in some embodiments may be referred to as a suture or suture device), and markings at regular length intervals to visually indicate length. For example, the markings can be a regular or specified length intervals along a length of the suture, providing an indication of a length of one or more portions of the suture.

According to some embodiments, the markings are darker in contrast to the flexible elongate member (e.g. suture). In some cases, the markings are lighter in contrast to the flexible elongate member (e.g. suture). In some cases, a marking can have a first visual characteristic (e.g. color, shading, pattern, or the like) and the flexible elongate member or suture can have a second visual characteristic (e.g. color, shading, pattern, or the like) that is different from the first visual characteristic. In some cases, the visual characteristic is optically detectable by the unaided human eye. In some cases, the visual characteristic is optically detectable by an aided human eye (e.g. aided with a magnification device, a light filtering device, a light enhancement device, a light detection device, or the like). In some cases, the markings have a length, such as a predetermined or known length. In some cases, the length of the markings are equal to the length of the interval between the markings. In some cases, the length of the markings and the length of the interval between the markings is 5 mm. In some cases, the length of the markings and the length of the interval between the markings is 2.5 mm. In some cases, the markings have a length, and the length of the markings are less than the length of the interval between the markings. In some cases, the markings are comprised of a radiopaque material for visualization on fluoroscopy, x-ray, and/or ultrasound. In some cases, the markings contain sequential identifiers. In some cases, the sequential identifiers include a number of contrasting bands corresponding to number in the marking sequence. In some cases, the markings are created by contrasting filament. In some cases, the contrasting filament is visually or optically distinguishable from other portions of the suture. In some cases, the markings are created by embroidery with a contrasting filament. In some cases, the markings are created by externalizing a contrasting core filament. In some cases, the markings are created by externalizing one or more portions of a contrasting core filament. In some cases, the markings are created by one filament of the external braid being a contrasting color.

In another aspect, embodiments of the present invention encompass methods for creating one or more markings on a suture. Exemplary methods can include submersing regular intervals of the suture in a colorant. In some cases, the colorant is a dye.

In another aspect, embodiments of the present invention encompass methods for creating one or more markings on a suture using a masking technique. Exemplary methods can include masking areas on the suture at regular intervals, and applying the colorant.

In another aspect, embodiments of the present invention encompass methods for creating one or more markings on a suture using a pad printing technique. Exemplary methods can include pad printing the markings.

### BRIEF DESCRIPTION OF THE DRAWINGS

Inventive features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A and 1B illustrate a suture with measurement units according to embodiments of the present invention.

FIGS. 2A and 2B illustrate a suture with measurement units according to embodiments of the present invention.

FIGS. 3A, 3B, 3C, and 3D illustrate sutures with measurement units according to embodiments of the present invention.

FIG. 4 illustrates a suture with measurement units according to embodiments of the present invention.

FIG. 5 illustrates a method to manufacture a suture with measurement units according to embodiments of the present invention.

FIGS. 6A and 6B illustrate a suture with measurement units according to embodiments of the present invention.

FIG. 7 illustrates a suture with measurement units according to embodiments of the present invention.

FIG. 8 illustrates a suture with measurement units according to embodiments of the present invention.

FIGS. 9A and 9B illustrate a method to manufacture a suture with measurement units according to embodiments of the present invention.

FIGS. 10A and 10B illustrate a method to manufacture a suture with measurement units according to embodiments of the present invention.

FIG. 11 illustrates a method to manufacture a suture with measurement units according to embodiments of the present invention.

### DETAILED DESCRIPTION

Specific embodiments of the disclosed sutures and methods of use and manufacture will now be described with reference to the drawings. Nothing in this detailed description is intended to imply that any particular component, feature, or step is essential to embodiments of the invention. Sutures disclosed herein can be used for attaching tissue to a variety of tissues, implants or instruments in a variety of patient treatment sites.

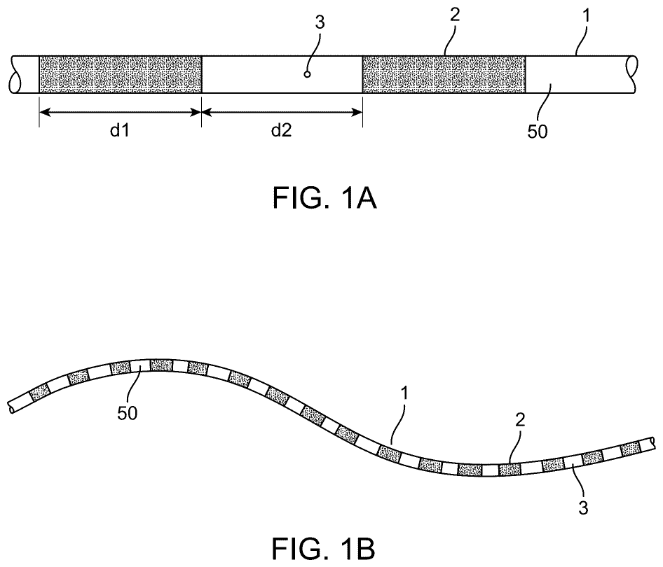

FIGS. 1A and 1B depict aspects of a suture measurement device 1, according to embodiments of the present invention. In some cases, a suture measurement device or suture measuring device can include a suture 50 and one or more markings or visually distinguishable regions. As shown there are dark regions 2 of a specific length (d1) and light regions 3 of the same length (d2). As shown here, the dark regions or markings can be disposed at regular length intervals having an interval length (e.g. d2) between the dark regions or markings. In some cases, instead of dark and light regions, a device can include some regions of a first color and some regions of a second color that is different from the first color. In different embodiments, the length of the dark and light regions do not have to be equal, they can be of different lengths. For example, the length of the dark region could be 5 mm and the length of the light region could be 5 mm. In some cases, the length of the dark region could be 2.5 mm and the length of the light region could be 2.5 mm. With this arrangement the surgeon could quickly measure length in increments of 2.5 mm. The dark regions 2 can be added to suture 50 by laser marking, dyeing, painting, coating or other similar methods. The substrate suture 50 could be monofilament or braided and could consist of absorbable or non-absorbable materials. In other embodiments, the dark regions 2 could also include a radio-opaque dye (such as Barium Sulfate) to allow the surgeon to make measurements on x-ray or fluoroscopy. In some cases, markings such as dark regions can enable magnification. In some cases, markings can enable the surgeon to determine if the tendon (e.g. to which a suture is attached) moves under dynamic x-ray or fluoroscopy techniques. In some cases, markings can enable a surgeon to assess kinematics. In some cases, a surgeon may line the spine or other anatomical feature of a patient, and then move or flex the spine or anatomy.

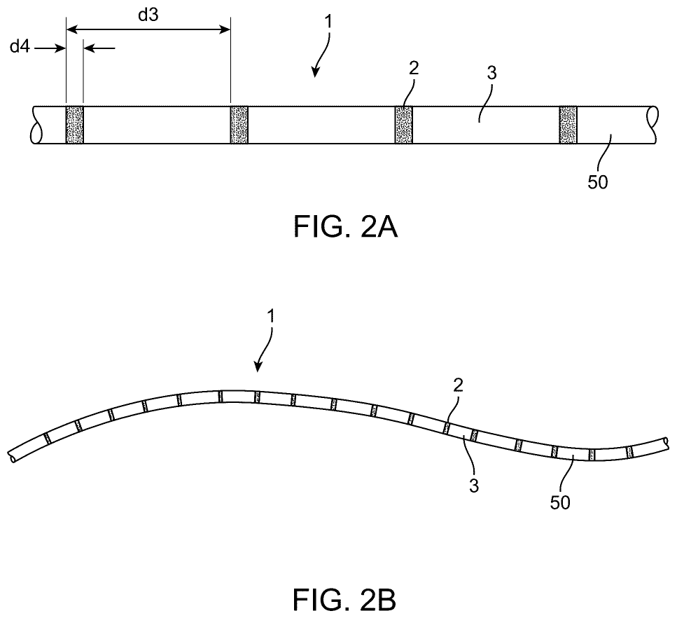

FIGS. 2A and 2B depict aspects of another embodiment of a suture measurement device 1. As shown in these figures the dark region 2 is a length (e.g. d4) equal to or less than the length (e.g. d3) of the light region 3. The distance between the dark regions could be at a regular interval such as 5 mm along the length of the suture 50. As shown here, the dark regions or markings can be disposed at regular length intervals having an interval length (e.g. d3) between the dark regions or markings. In some cases, the markings or dark regions have a length, and the length of the markings is less than the length of the interval between the markings. In some cases, the markings or dark regions have a length, and the length of the markings is greater than the length of the interval between the markings. In some cases, the markings or dark regions have a length, and the length of the markings is equal to the length of the interval between the markings.

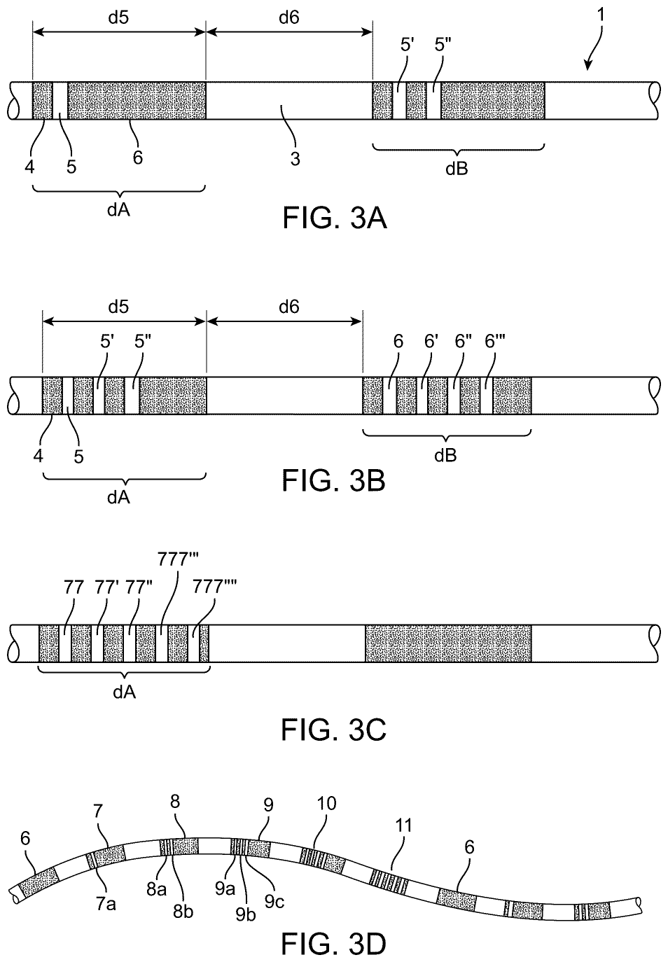

FIGS. 3A, 3B, 3C, and 3D depict other embodiments of a suture measurement device 1. In exemplary embodiments additional light regions 5 (e.g. within a dark region dA) have been added to identify the length. The identifier light region 5 allows the surgeon/user to identify the number of length units at this point. FIG. 3A has one identifier light region 5 that splits the dark region dA into two dark regions 4 and 6. The length is indicated d5 is the total length from the start of dark region 4 to the end of dark region 6. In this embodiment of the invention, the light region 3 length d6 is equal to the length indicated by the dark regions d5. In other embodiments these lengths (d5) and (d6) may be of different lengths. FIG. 3A also shows the next dark region dB consisting of two identifier light regions 5′ and 5″ to indicate that this is the second marker. As shown here, the dark regions or markings can be disposed at regular length intervals having an interval length (e.g. d6) between the dark regions or markings.

FIG. 3B illustrates a dark region dA with 3 identifier light regions 5, 5′, and 5′ on the left of the image and a dark region dB with 4 identifier light regions 6, 6′, 6″, 6′″, on the right of the image. FIG. 3C illustrates a dark region dA with 5 identifier light regions 77, 77′, 77″, 77′″, 77″″ on the left of the image. FIG. 3D illustrates a suture measuring device with length increments and identifier light regions to communicate length to the surgeon/user. From left to right, the first dark region 6 has zero identifier light regions, the second dark region 7 has one identifier light region 7a, the third dark region 8 has two identifier light regions 8a, 8b, the fourth dark region 9 has three identifier light regions 9a, 9b, 9c, the fourth dark region 10 has four identifier light regions, and the fifth dark region 11 has five identifier light regions. In this embodiment the pattern repeats itself and the next sixth dark region 6 has zero identifier light regions. In this embodiment, the identifier light regions are shown as circumferential rings. In other embodiments these identifier regions could be circles or other shapes to communicate the order of markings. Hence, markings can include or contain sequential identifiers, and such sequential identifiers can include a number of contrasting bands corresponding to number in the marking sequence (e.g. a single contrasting band 7a corresponding to the first number in the sequence, two contrasting bands 8a, 8b corresponding to the second number in the sequence, and so on).

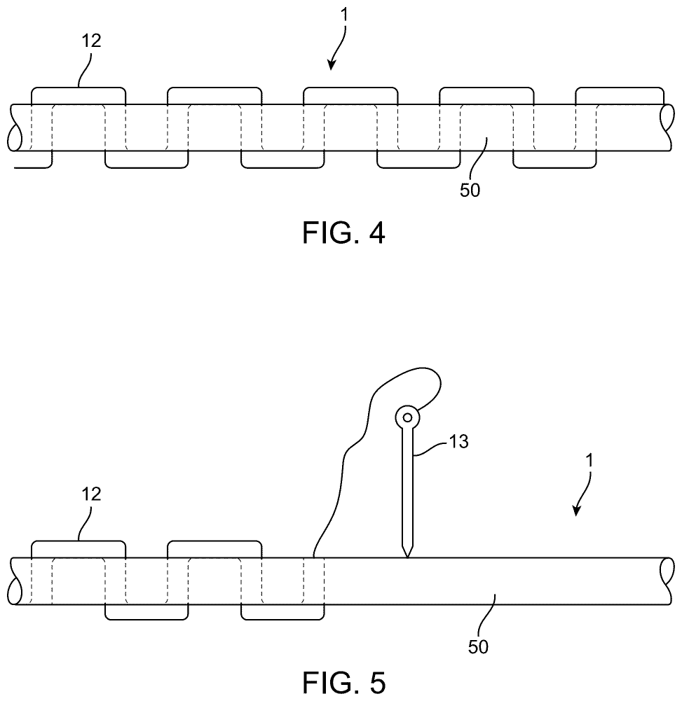

FIG. 4 illustrates yet another embodiment of a suture measuring device 1. In this embodiment the length units are indicated by a marking filament 12 that passes through the suture 50 and extends substantially parallel to the long axis of the suture 50 for the given length unit and then passes through the suture 50. This pattern repeats itself along the length of the suture 50. In this embodiment, the suture 50 could be constructed with braided filament and at least one filament marking filament 12 could indicate length. In additional embodiments, the suture may have multiple marking filaments 12. Additionally, in alternative embodiments the multiple marking filaments may indicate different length intervals.

FIG. 5 shows a manufacturing method to pass the marking filament 12 through the suture 50 using a needle 13 to produce the suture measuring device 1. This process of passing the needle through the suture 50 can be done manually (by hand) or automated with a machine. The automated machine could be put down stream from the braiding machine.

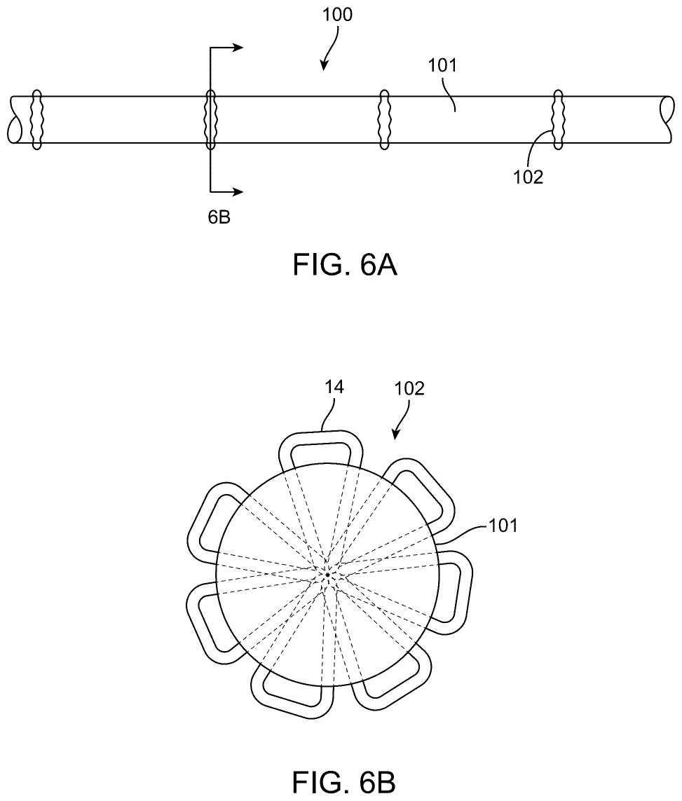

FIG. 6A illustrates another embodiment of the suture measuring device 100 where the length markings 102 are made from embroidery of a contrasting filament. The embroidered length markings would occur at regular length intervals (e.g. along a length of the suture 101).

FIG. 6B shows a cross section of the embroidery at the length marking 102. The filament 14 used to embroider the length marking passes through suture 101. This process is repeated several times around the circumference of the suture 101 to create the length marking 102. The filament 14 could be of a similar material to that of the suture 101 or a different material. In an alternative embodiment, the filament could be comprised of a radiopaque material. In yet another embodiment, the filament could be comprised of an absorbable material.

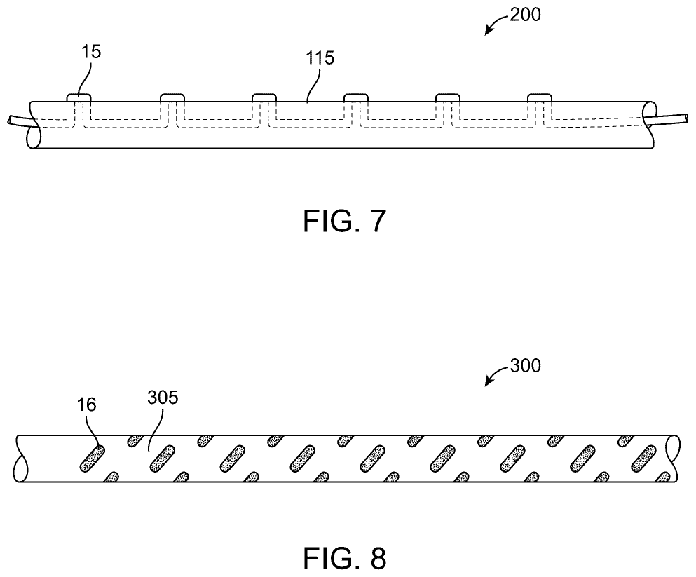

FIG. 7 illustrates another embodiment of a suture measuring device 200 where at least one filament from the core 15 of a braided suture exits the external braid and re-enters the external braid to join the remaining core filaments of the suture 115. The marking filament from the core 15 could be of a different shade or color from the rest of the suture 115 to provide visual contrast to the user/surgeon.

FIG. 8 shows another embodiment of a suture measuring device 300. One filament of the external braid 16 is made of a contrasting color and spaced at regular intervals along the suture 305 to provide the length measurements. Hence, in some cases, a marking can be created by one filament of an external braid being a contrasting color (e.g. in comparison to other braids or elements of the suture).

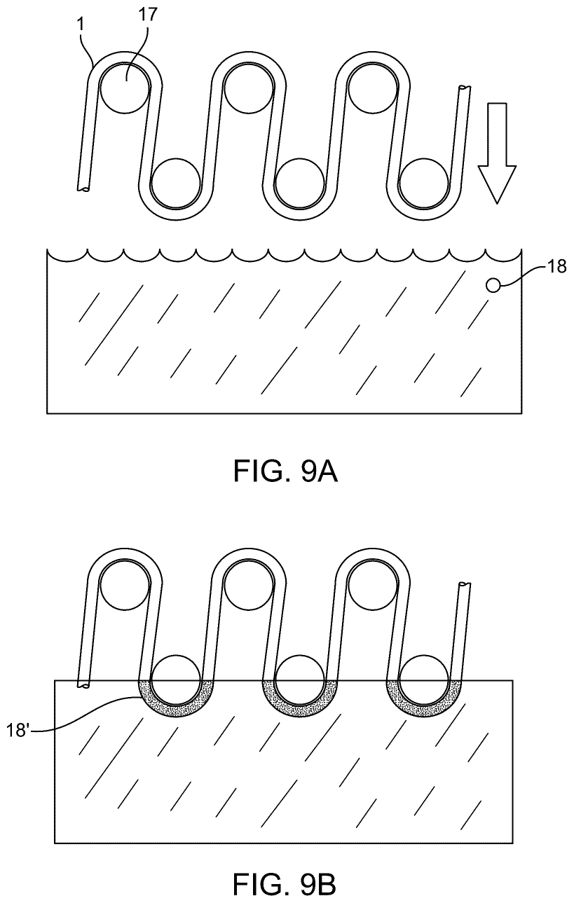

FIG. 9A illustrates a method of creating the markings (e.g. dark regions) during manufacturing. The suture 1 is passed around a fixture 17 to orient the suture to create the regular interval of markings. The fixture sits above a tank of colorant 18. The colorant could be food grade dye or other biocompatible coloring agents. The fixture and colorant tank could be inserted down the manufacturing line to further facilitate automation of the marking process in line with the braiding machine. The fixture 17 could be created with a multitude of rollers or pins to facilitate automation.

FIG. 9B further illustrates a method for creating the markings. The figure shows the suture 1 inserted into the tank of colorant 18. The suture contacts the colorant in the regions needed to create the length markings 18′ at regular intervals.

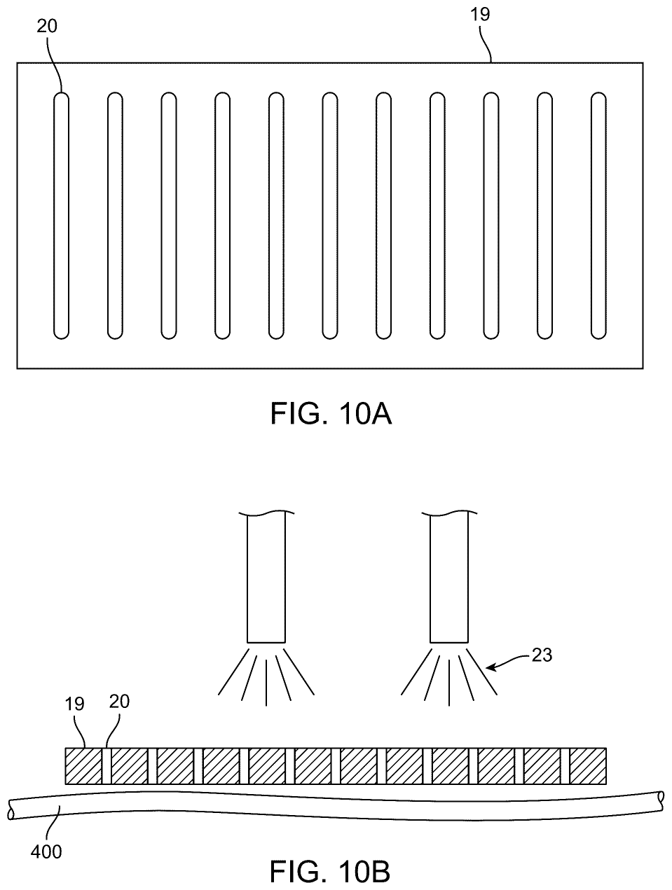

FIG. 10A shows a top view of another method to create the markings on a suture using a masking fixture 19. The masking fixture 19 has openings 20 at regular intervals where the markings will be applied to the suture. FIG. 10B shows a side view of the masking fixture 19. The suture 400 is positioned below the masking fixture and the colorant 23 (e.g. dye, paint, or the like) is sprayed on top of the masking fixture 19. The openings 20 allow the colorant to be applied to the suture 400 at the regular intervals desired (e.g. to unmasked or uncovered locations along the suture). This process would allow for automation in line with the suture braider or could be performed as a second operation. If performed in a second operation, multiple sutures could be sprayed or colored at the same time.

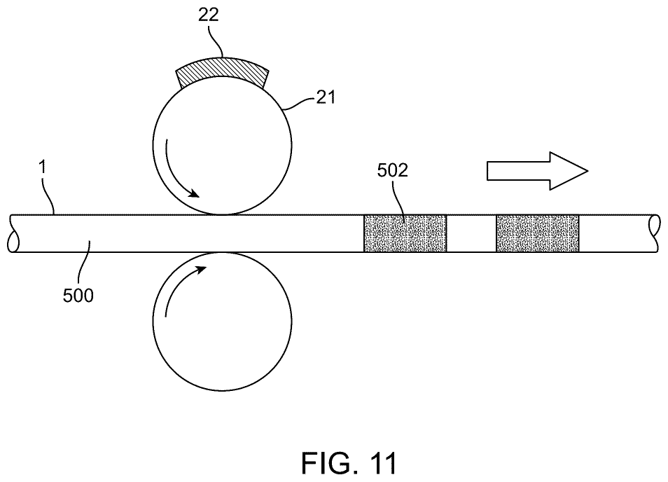

FIG. 11 illustrates another method to apply markings 502 to a suture 500. This method has a pair of rollers 21 where the suture 500 passes between. At least one of the rollers has at least one pad 22 for colorant transfer to the suture 500 to produce the suture measuring device 1. The roller method could be configured to be applied downstream from the braiding machine and allow a continuous method of marking application. The roller could have multiple pads 22.

Although the methods and apparatuses are described herein in terms of various exemplary embodiments and implementations, it should be understood that the various features, aspects and functionality described in one or more of the individual embodiments are not limited in their applicability to the particular embodiment with which they are described, but instead might be applied, alone or in various combinations, to one or more of the other embodiments of the disclosed method and apparatus, whether or not such embodiments are described and whether or not such features are presented as being a part of a described embodiment. Thus the breadth and scope of the claimed invention should not be limited by any of the herein-described embodiments.

Terms and phrases used in this document, and variations thereof, unless otherwise expressly stated, should be construed as open-ended as opposed to limiting. As examples of the foregoing: the term “including” should be read as meaning “including, without limitation” or the like, the term “example” is used to provide exemplary instances of the item in discussion, not an exhaustive or limiting list thereof, the terms “a” or “an” should be read as meaning “at least one,” “one or more,” or the like, and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that might be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases might be absent. Any use of the term “assembly” does not imply that the components or functionality described or claimed as part of the module are all configured in a common package. Indeed, any or all of the various components of a module, whether control logic or other components, might be combined in a single package or separately maintained and might further be distributed across multiple locations.

Although the description herein contains significant detail in relation to certain embodiments, which in some cases may be preferred embodiments, it should not be construed as limiting the scope of the invention but rather as providing illustrations of certain exemplary or preferred embodiments.

Embodiments of the present invention encompass kits having one or more components of a device or system as disclosed herein. In some embodiments, the kit includes one or more device or system components, along with instructions for using the component(s) for example according to any of the methods disclosed herein.

All features of the described systems and devices described herein can be applicable to the described methods mutatis mutandis, and vice versa.

In addition, each reference provided herein is incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A suture measuring device, comprising:
a flexible elongate member; and
markings at regular length intervals along the flexible elongate member to visually indicate length, the regular length intervals having an interval length between the markings.

2. The suture measuring device according to claim 1, wherein the flexible elongate member comprises a suture.

3. The suture measuring device according to claim 1, wherein the markings are darker in contrast to the flexible elongate member.

4. The suture measuring device according to claim 1, wherein the markings are lighter in contrast to the flexible elongate member.

5. The suture measuring device according to claim 1, wherein the markings have a length, and wherein the length of the markings is equal to the length of the interval between the markings.

6. The suture measuring device according to claim 5, wherein the length of the markings and the length of the interval between the markings is 5 mm.

7. The suture measuring device according to claim 5, wherein the length of the markings and the length of the interval between the markings is 2.5 mm.

8. The suture measuring device according to claim 1, wherein the markings have a length, and wherein the length of the markings is less than the length of the interval between the markings.

9. The suture measuring device according to claim 1, wherein the markings comprise a radiopaque material
for visualization by a member selected from the group consisting of a fluoroscopy visualization technique, an x-ray visualization technique, and an ultrasound visualization technique.

10. The suture measuring device according to claim 1, wherein the markings comprise sequential identifiers.

11. The suture measuring device according to claim 10, wherein the sequential identifiers comprise of a number of contrasting bands corresponding to a number in the marking sequence.

12. The suture measuring device according to claim 1, wherein the markings are created by contrasting filament.

13. The suture measuring device according to claim 1, wherein the markings are created by embroidery with a contrasting filament.

14. The suture measuring device according to claim 1, wherein the markings are created by externalizing a contrasting core filament.

15. The suture measuring device according to claim 1, wherein the markings are created by one filament of an external braid being a contrasting color.

16. A method for creating markings on a suture, the method comprising:
submersing regular intervals of the suture in a colorant.

17. The method according to claim 16, wherein the colorant comprises a dye.

18. A method for creating markings on a suture, the method comprising:
masking areas on the suture at regular intervals; and
applying the colorant to unmasked areas on the suture.

19. A method for creating markings on a suture, the method comprising:
pad printing the markings on the suture.
